(19) United States
(12) Patent Application Publication
(10) Pub. No.: US 2021/0183526 A1
(43) Pub. Date: Jun. 17, 2021

(54) UNSUPERVISED TAXONOMY EXTRACTION FROM MEDICAL CLINICAL TRIALS

(71) Applicant: Trialmatch.me, Inc. d/b/a/Trialjectory, Stamford, CT (US)

(72) Inventors: Tzvia Bader, Closter, NJ (US); Guy Gildor, Herzelia (IL)

(21) Appl. No.: 17/124,216

(22) Filed: Dec. 16, 2020

Related U.S. Application Data
(60) Provisional application No. 62/948,696, filed on Dec. 16, 2019.

(51) Int. Cl. G16H 50/70; G16H 10/20; G16H 70/20; G16H 70/60; G16H 50/20; G16H 10/60; G06F 16/906; G06F 16/903

(52) U.S. Cl. CPC G16H 50/70; G16H 10/20; G16H 70/20; G16H 70/60; G16H 40/20; G16H 10/60; G06F 16/906; G06F 16/90344; G16H 50/20

(57) ABSTRACT

Unsupervised taxonomy extraction from medical clinical trials for supplementing a keyword mapping to disease conditions is provided. One or more corpus of clinical trial descriptions is read. A list of disease conditions is read. A plurality of categories of clinical trials is determined. A frequency of occurrence for each of one or more repeated terms from each category of clinical trials is determined. A set of category-specific repeated terms is determined. A set of new terms is determined. A plurality of vectors for each new term in the set of new terms is determined where each vector for a particular new term corresponding to the one or more associated keyword. One or more of the new terms in the set of new terms is selected based on the vectors. Each of the selected new terms is mapped to a disease condition thereby generating a supplemented list of disease conditions.

### CROSS-REFERENCE TO RELATED APPLICATION

This application claims the benefit of U.S. Provisional Patent No. 62/948,696, filed on Dec. 16, 2019, which is hereby incorporated by reference herein in its entirety.

### BACKGROUND

Embodiments of the present disclosure relate to analytics for clinical trial criteria, and more specifically, to unsupervised taxonomy extraction from medical clinical trials.

### BRIEF SUMMARY

According to embodiments of the present disclosure, methods of and computer program products for unsupervised taxonomy extraction from medical clinical trials are provided.

A method is provided where one or more corpus is read. Each of the one or more corpus has a plurality of clinical trial descriptions. A list of disease conditions is read. The list of disease conditions has one or more disease condition mapped to one or more associated keyword. A plurality of categories of clinical trials is determined based on the plurality of clinical trial descriptions and the list of disease conditions. A frequency of occurrence for each of one or more repeated terms is determined from each category of clinical trials. A set of category-specific repeated terms having a frequency of occurrence greater than a predetermined threshold is determined. Whether each repeated term in the set of category-specific repeated terms is not present in a predetermined medical taxonomy is determined to thereby identify a set of new terms. A plurality of vectors for each new term in the set of new terms is determined. Each vector for a particular new term corresponds to the one or more associated keyword. Based on the plurality of vectors, one or more of the new terms in the set of new terms is selected. Each of the selected new terms is mapped to a disease condition thereby generating a supplemented list of disease conditions.

A system is provided including a data store and a computing node including a computer readable storage medium having program instructions embodied therewith. The program instructions are executable by a processor of the computing node to cause the processor to perform a method where one or more corpus is read. Each of the one or more corpus has a plurality of clinical trial descriptions. A list of disease conditions is read. The list of disease conditions has one or more disease condition mapped to one or more associated keyword. A plurality of categories of clinical trials is determined based on the plurality of clinical trial descriptions and the list of disease conditions. A frequency of occurrence for each of one or more repeated terms is determined from each category of clinical trials. A set of category-specific repeated terms having a frequency of occurrence greater than a predetermined threshold is determined. Whether each repeated term in the set of category-specific repeated terms is not present in a predetermined medical taxonomy is determined to thereby identify a set of new terms. A plurality of vectors for each new term in the set of new terms is determined. Each vector for a particular new term corresponds to the one or more associated keyword. Based on the plurality of vectors, one or more of the new terms in the set of new terms is selected. Each of the selected new terms is mapped to a disease condition thereby generating a supplemented list of disease conditions.

A computer program product is provided for supplementing a keyword mapping to disease conditions based on clinical trial descriptions. The computer program product includes a computer readable storage medium having program instructions embodied therewith. The program instructions are executable by a processor to cause the processor to perform a method where one or more corpus is read. Each of the one or more corpus has a plurality of clinical trial descriptions. A list of disease conditions is read. The list of disease conditions has one or more disease condition mapped to one or more associated keyword. A plurality of categories of clinical trials is determined based on the plurality of clinical trial descriptions and the list of disease conditions. A frequency of occurrence for each of one or more repeated terms is determined from each category of clinical trials. A set of category-specific repeated terms having a frequency of occurrence greater than a predetermined threshold is determined. Whether each repeated term in the set of category-specific repeated terms is not present in a predetermined medical taxonomy is determined to thereby identify a set of new terms. A plurality of vectors for each new term in the set of new terms is determined. Each vector for a particular new term corresponds to the one or more associated keyword. Based on the plurality of vectors, one or more of the new terms in the set of new terms is selected. Each of the selected new terms is mapped to a disease condition thereby generating a supplemented list of disease conditions.

A method is provided where a plurality of categories of clinical trials is read. Each category of clinical trials corresponds to a unique disease condition and has a plurality of associated keywords. A new medical term is received. The new medical term is not present in any of the plurality of categories of clinical trials. For each category in the plurality of categories of clinical trials, the new medical term is compared to each associated keyword to determine for each new medical term and associated keyword pair: a distance metric between the new medical term and associated keyword, double occurrences of the new medical term and associated keyword, and triple occurrences of the new medical term, associated keyword, and an additional medical term. A vector magnitude is determined for each new medical term and associated keyword pair.

A system is provided including a data store and a computing node including a computer readable storage medium having program instructions embodied therewith. The program instructions are executable by a processor of the computing node to cause the processor to perform a method where a plurality of categories of clinical trials is read from the datastore. Each category of clinical trials corresponds to a unique disease condition and has a plurality of associated keywords. A new medical term is received. The new medical term is not present in any of the plurality of categories of clinical trials. For each category in the plurality of categories of clinical trials, the new medical term is compared to each associated keyword to determine for each new medical term and associated keyword pair: a distance metric between the new medical term and associated keyword, double occurrences of the new medical term and associated keyword, and triple occurrences of the new medical term, associated keyword, and an additional medical term. A vector magnitude is determined for each new medical term and associated keyword pair.

A computer program product for determining a vector between an unknown medical term and a known medical term, the computer program product comprising a computer readable storage medium having program instructions embodied therewith, the program instructions executable by a processor to cause the processor to perform a method where a plurality of categories of clinical trials is read. Each category of clinical trials corresponds to a unique disease condition and has a plurality of associated keywords. A new medical term is received. The new medical term is not present in any of the plurality of categories of clinical trials. For each category in the plurality of categories of clinical trials, the new medical term is compared to each associated keyword to determine for each new medical term and associated keyword pair: a distance metric between the new medical term and associated keyword, double occurrences of the new medical term and associated keyword, and triple occurrences of the new medical term, associated keyword, and an additional medical term. A vector magnitude is determined for each new medical term and associated keyword pair.

### BRIEF DESCRIPTION OF THE SEVERAL VIEWS OF THE DRAWINGS

FIG. 1 depicts an exemplary system for unsupervised taxonomy extraction for medical clinical trials according to embodiments of the present disclosure.

FIG. 2 depicts an exemplary method of unsupervised taxonomy extraction for medical clinical trials according to embodiments of the present disclosure.

FIG. 3 depicts an exemplary Map/Reduce process for counting words according to embodiments of the present disclosure.

FIG. 4 depicts an exemplary process for determining repeated words in categories of clinical trials according to embodiments of the present disclosure.

FIG. 5 depicts a computing node according to an embodiment of the present disclosure.

### DETAILED DESCRIPTION

Clinical trials are research studies that are used to test new and promising techniques to diagnose, prevent, or treat a disease (such as cancer). Clinical trials can be used to learn if a new treatment is more effective or has less harmful side effects than the standard treatment.

At present, various clinical trial registries are maintained by different healthcare organization. For example, in the US, such a registry is maintained by the US National Library of Medicine and is accessible at ClinicalTrials.gov. A similar registry is available through the UK Clinical Trials Gateway (UKCTG).

However, there is no systematic way to match patients to clinical trials. As a result, a patient may not discover relevant trials in a timely manner. In addition, even if a patient knows where to look, the trial descriptions are not easy to interpret, and it is frequently unclear whether a given patient is eligible for a given trial. This is particularly challenging for oncology trials, where there is extreme complexity and volume of trials.

Moreover, clinical trial descriptions may not be written using a standardized lexicon of medical terminology and/or medical taxonomies. This may create problems when matching patients to clinical trials because using a known medical lexicon and/or known medical taxonomies to determine which disease(s) to which the clinical trial is directed likely will not recognize words outside of the lexicon or taxonomy.

To address these and other shortcomings of the prior art, the present disclosure provides systems and methods suitable for matching patients to clinical trials in a systematic, accurate, and automated way. In particular, the present disclosure provides for unsupervised taxonomy extraction for medical clinical trials. Additionally, the present disclosure provides systems and methods for updating and/or supplementing a medical taxonomy (and/or lexicon) with new terms that are related to a specific disease condition thereby improving patient-trial matching and, ultimately, patient outcomes.

In various embodiments, medical information of a patient is compared to the enrollment criteria of available trials, and matching trials are recommended. Matching trials may be ranked based on a variety of criteria, including compatibility with a patient's medical history, distance, trial type, or timing. For example, interventional trials may be ranked above observational trials, or trials without placebos may be ranked above trials that include placebos. In another example, trials located closer to a patient's home are ranked above trials further away. The list can be filtered by additional criteria, such as location, trial phase, and other non-clinical parameters. In addition, a medical profile may be matched with other viable treatment options outside of clinical trials (e.g., drugs approved for other indications).

In some embodiments, a medical questionnaire is provided to patients seeking treatment. In some embodiments, patient medical records are read directly from electronic health records. For example, in some embodiments, a web app is provided that allows patients and their physicians to self-build their clinical profile by filling out an adaptive questionnaire that includes information about disease characteristics, treatment history and overall health. Each completed profile may then be matched with the eligibility criteria of available clinical trials to produce a short list of relevant matched trials. A medical questionnaire is particularly useful for patients and community oncology clinics that don't have robust EMR solutions and no tools to identify and match patient to clinical trial. Medical questionnaires also enable bypass of EMR data and the inherent inconsistencies and challenges in data integration.

However, in some embodiments, EMR data are used to provide patient profiles without the need for completion of a questionnaire.

An electronic health record (EHR), or electronic medical record (EMR), may refer to the systematized collection of patient and population electronically-stored health information in a digital format. These records can be shared across different health care settings and may extend beyond the information available in a PACS. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long-term changes in a patient. Population-based studies of medical records may also be facilitated by the widespread adoption of EHRs and EMRs.

In order to provide appropriate clinical trial matching, the present disclosure provides methods for reading, analyzing, and structuring clinical trial description data. Such methods may be deployed in concert with methods for EMR structuring.

In various embodiments, a natural language processing (NLP) engine is provided that creates a rich medical taxonomy through unsupervised learning. Clinical trial descriptions do not require consistency in the use of medical terms, and thus are highly variable in content. Additionally, due to the innovative nature of clinical trials, newly coined and defined medical terms and expressions are constantly added to the database. Accordingly, various embodiments are able to avoid reliance on a given or pre-set taxonomy (e.g., provided from external sources). Newly coined medical terms are identified, extracted, and understood the from the medical text context. This NLP engine combines morphology and sentence structure analysis with content analysis, using frequency and distance to create a medical semantic network on the fly. In addition to creating this medical taxonomy, unsupervised clustering analysis is applied for concept extraction to transform the tagged clinical trial descriptions into a vector of trial exclusion and inclusion criteria. By creating a metric (e.g., a distance function) between the different textual representation of these criteria, joint attributes may be identified that might have different values but represent similar properties of patients. By clustering these together, the terms are unified into a more stable list of attributes.

In various embodiments, deep learning is applied to optimize the match between a patient's disease profile and trial eligibility criteria. Using a neural network, trials may be identified that match with each patient profile.

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

Alternative machine learning solutions may include analysis of existing EMR data to identify patients with profiles that fit a given trial. Such a solution is trial-specific, that is it seeks to establish a one-to-one relationship between a patient and a certain trial. This approach addresses the issue that EMR data is partly unstructured, may appear in PDF format, and/or can be hand-written. Relevant EMR data may be spread through different systems, and may be incomplete or missing. Solutions that start from EMR data try to identify general medical attributes within records are susceptible to sparse data matrices and are less dynamic with regard to medical cutting-edge protocols and drugs.

As set out below, in various embodiments, all recruiting trials are analyzed, not just a given trial under consideration. In various embodiments, clinical trial descriptions are analyzed using unsupervised learning. Accordingly, only medical attributes that are relevant to trial criteria are identified, allowing systems that need fewer attributes and result in denser data matrices.

The approaches described herein are applicable to any dataset of clinical trial or pharmaceutical indications. For example, the present disclosure is applicable in the field of oncology, such as to trials for breast cancer, colon cancer, bladder cancer, melanoma, or myelodysplastic syndromes (MDS; often called preleukemia).

In various embodiments, an engine is provided that reads all unstructured treatment descriptions from a clinical trial dataset and extracts the data that is relevant to a given patient. The information is clustered, classified, and standardized, creating a dataset highlighting the patient attributes that clinical trials are looking for. Patients are then matched to clinical trials through self-reported dynamic questionnaire answers or EMR. In various embodiments, a user can then filter matched trials and share the information with their physicians (e.g., oncologists) to move forward in the process if appropriate.

In various embodiments, unstructured text describing medical clinical trials inclusion and exclusion criteria are converted into structured data. Such structured data provides logical conditions within a structured space of distinctive and normalized medical conditions. In various embodiments, this entails identifying, extracting, normalizing, and interpreting the specific medical terms used in these texts in their specific context.

One approach to this task is to use pre-defined dictionaries and taxonomies of medical terms. Various such taxonomies are created manually by experts in appropriate domains. These taxonomies are updated on a quarterly, semi-annual, or annual basis. Reliance on such taxonomies is problematic for clinical trial analysis, as clinical trials often coin new terms for drugs, treatments, and even disease types. Accordingly, waiting for updates to predetermined taxonomies severely limits the applicability of taxonomy-reliant NLP techniques to clinical trial data. Accordingly, the present disclosure provides methods to automatically identify new terms and automatically interpret and provide context.

In various embodiments, the text of clinical trials is analyzed to identify repeating terms that are new to preexisting taxonomies (e.g., medical dictionaries). Probabilities are assigned to these terms, indicating to which parts of the medical semantic tree they belong, based on the context in which they appear. For example, a new term that appears as part of a list of known chemotherapy drugs that are used to treat breast cancer, it may be assumed with high probability that the new term is also such a drug.

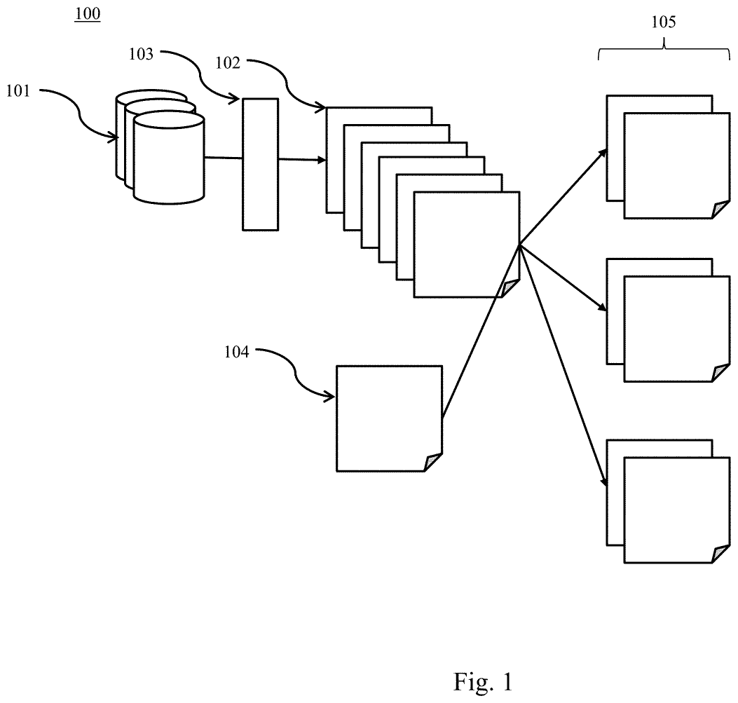

In FIG. 1, an exemplary system 100 for unsupervised taxonomy extraction for medical clinical trials is illustrated according to embodiments of the present disclosure. As shown in FIG. 1, the system 100 includes one or more corpus 101 of clinical trials related to a plurality of clinical trials 102. In various embodiments, each of the one or more corpus 101 may include structured or unstructured data relating to one or more clinical trials. In various embodiments, the one or more corpus 101 of clinical trials may be accessed via an API 103 to retrieve data on a plurality of clinical trials 102. In various embodiments, where an API is not provided, the plurality of clinical trials 102 may be extracted from the one or more corpus 101 of clinical trials directly via a direct method. For example, data regarding clinical trials may be stored in unstructured text. Unstructured data, or data that are structured according to a schema that is inconsistent with the intended use, may require additional processing to determine the attributes of interest for a given use case. To address this, in various embodiments, the direct method may include application of an artificial neural network. In various embodiments, the direct method may include a template-based approach. A template-based approach may be used where a corpus 101 uses a standardized format for structuring clinical trial data. In various embodiments, the system 100 may access one or more list of medical conditions 104 to thereby categorize the clinical trial descriptions extracted from the clinical trial corpus 101.

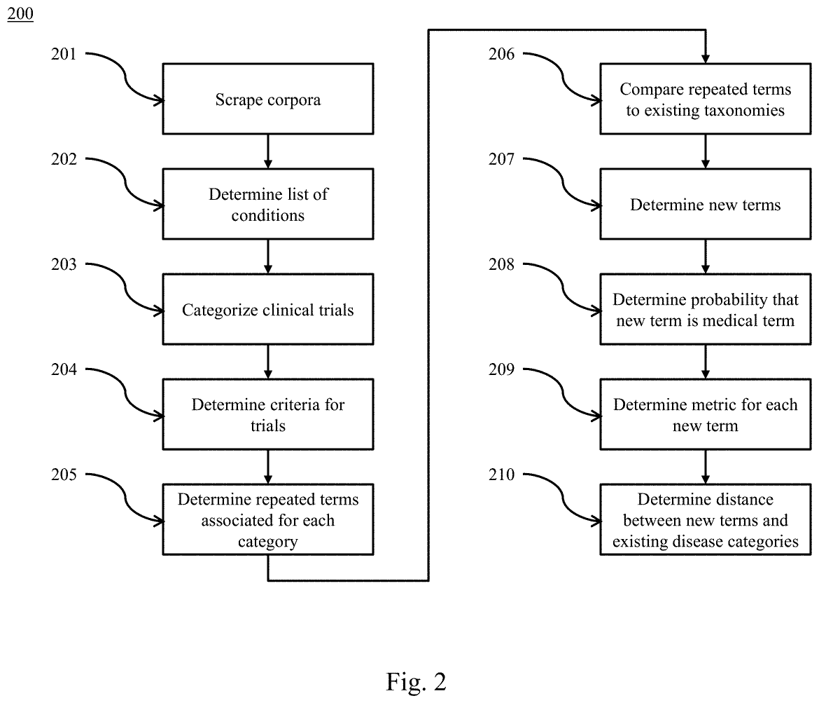

In FIG. 2, an exemplary method of unsupervised taxonomy extraction for medical clinical trials is illustrated according to embodiments of the present disclosure.

At 201, at least one corpus 101 of clinical trials is scraped to extract data related to a plurality of clinical trials 102. Exemplary corpora include ClinicalTrials.gov (available at https://clinicaltrials.gov/), EU Clinical Trials Register (available at https://www.clinicaltrialsregister.eu/), CenterWatch (available at https://www.centerwatch.com/), Chinese Clinical Trial Registry (available at http://www.chictr.org.cn/index.aspx), National Cancer Institute (available at https://www.cancer/gov/about-cancer/treatment/clinical-trials/search), and the National Institutes of Health (available at https://www.nih.gov/health-information). In various embodiments, the data are collected using an API 103 exported by providers of corpora 101, while in some embodiments an external scraping tool is used.

In some embodiments, the data provided is structured data. In some embodiments, the data are completely unstructured. In some embodiments the data are combination of structured and unstructured data. In various embodiments, the data may be provided in extensible markup language (XML). In various embodiments, the data may be provided as study metadata.

In various embodiments, clinical trial descriptions (e.g., protocols and/or publication) may be extracted. In various embodiments, the clinical trial descriptions may be extracted from official governmental clinical trial databases, such as AACT, NIH, EORTC and/or ChiCTR.

In various embodiments, the data may be collected from clinical trial protocols. In various embodiments, the data may be collected from one or more publication resulting from one or more clinical trial (e.g., clinical trial results). In various embodiments, reading data from one or both of these sources may provide users with, for example, a complete and more detailed profile of a clinical trial, a summary of a previous trial phase, and/or approved drugs.

In various embodiments, clinical trial descriptions may be extracted using natural language processing. In various embodiments, the natural language processing may include word segmentation (tokenization), parsing, stemming, morphological segmentation, named entity recognition, terminology extraction, sentiment analysis, negation detection, etc.

At 202, a list 104 of medical (e.g., disease) conditions is provided. In various embodiments, the list of medical conditions are extracted from one or more known medical taxonomies. In some embodiments, the list is generated manually, while in some embodiments the list is determined from a preexisting dictionary of conditions which may be manually or automatically generated. An exemplary list may contain a plurality of cancer types (e.g., MDS, AML, CRC, breast cancer) and/or additional conditions such as dementia, diabetes, or HIV. In various embodiments, the list of medical conditions may be stored locally in a local database. In various embodiments, the list of medical conditions may be stored in a database at a remote server (e.g., in the cloud) and accessed via the Internet.

At 203, the clinical trials 102 are categorized based on medical condition list 104, yielding a plurality of sets of clinical trials 105, providing a separate textual corpus for each condition in list 104. In some embodiments, each trial is associated with a disease name, for example through a disease name field in the clinical trial record. In some embodiments, the relevant condition is mentioned in a textual description of the clinical trial or other unstructured data.

In various embodiments, the clinical trials may be categorized into a hierarchy of disease conditions. For example, in a disease condition of cancer, the clinical trial may be categorized based on cancer types (e.g., “soft tissue”->“Leiomyosarcoma”, or “blood cancer”->“leukemia”). In various embodiments, the features used for this categorization are the both in the meta data and also extracted from the terms in the clinical trial description. In various embodiments, a single trial may belong to multiple different categories.

In some embodiments, the relevant disease name for a clinical trial is determined by keyword matching. In some embodiments, a fuzzy keyword matching is applied, allowing for variations in spelling or abbreviation. In some embodiments, semantic connections between different names for the same or similar conditions are used to determine a relevant disease name. In various embodiments, fuzzy keyword matching may identify non-exact matches of a target item, e.g., a disease condition. In various embodiments, a Damerau-Levenshtein distance function may be used for fuzzy string matching. In various embodiments, the minimum threshold for the fuzzy matching may be determined manually. In various embodiments, a target accuracy of the fuzzy keyword matching may be at least a 90% accuracy match (e.g., less than or equal to a 10% false positive rate). In various embodiments, a target accuracy of the fuzzy keyword matching may be at least a 95% accuracy match (e.g., less than or equal to a 5% false positive rate).

In various embodiments, all conditions mentioned in a clinical trial description (and/or publication) may be extracted. In various embodiments, any conditions mentioned in a clinical trial description (and/or publication) may be relevant to compare to a patient's medical condition for purpose of matching the patient to that clinical trial.

At 204, the medical criteria are identified for the trial participants within each clinical trial in a given category 105. Both inclusion and exclusion criteria are identified. In embodiments where clinical trial data is structured, inclusion and exclusion data may be pre-tagged in the record. In other embodiments, the inclusion and exclusion criteria may be identified by proximity to certain keywords in textual description of the clinical trial. In yet other embodiments, a neural network is trained to identify the portions of the clinical trial description containing the medical criteria. In various embodiments, inclusion and/or exclusion criteria may be identified by searching the clinical trial description for specific words (e.g., “inclusion”, “exclusion”). In various embodiments, inclusion and/or exclusion criteria may be identified using morphological extensions (such as “excluded”) and/or similar terms (“eligible”, “not eligible,” etc.).

In various embodiments, natural language processing (NLP) methods may be used to analyze the patient medical records. In various embodiments, the NLP methods may be similar to the NLP methods used to determine disease conditions in the clinical trials. In various embodiments, the patient's profile values may be extracted into a different metadata structure (schema) than the structure of the clinical trial eligibility criteria. In various embodiments, a patient medical profile may include four (4) types of attributes per patient medical profile: demographic, disease characteristics, treatment history and health conditions. Other suitable attributes may be incorporated into the patient medical profile as is known in the art.

At 205, repeated terms of extracted for each set 105. In particular, a frequency analysis is performed to identify terms that appear more frequently in a specific condition corpus compared to all the other condition corpora. In some embodiments, a score is computed according to Equation 1, where c corresponds to a given condition and t corresponds to a given term.

$${score}_{c} ( t ) = \frac{\frac{c {ount}_{c} ( t )}{{\sum }_{t} {count}_{c} ( t )}}{\frac{{\sum }_{c} {count}_{c} ( t )}{{\sum }_{c} {\sum }_{t} {count}_{c} ( t )}} \qquad Equation 1$$

At 206, the terms that are most characteristic of each category are compared to preexisting taxonomies to identify known medical terms. In some embodiments, terms are selected that have a score greater than one, and have a significant statistical count within the textual corpus for the category.

At 207, the new terms are identified by extracting those that do not appear in an existing taxonomy and have a high score. In some embodiments, a predetermined number of top scores are considered. These are considered to be new terms for further analysis.

At 208, for each new term, the probability to be a medical term is determined. In various embodiments, a medical term includes any word or phrase having clinical importance, e.g., a genetic mutation, biomarker, or drug name. In some embodiments, a neural network is pretrained on existing terms from existing taxonomies. In such embodiments, the network is configured to receive as input a term and its surrounding context (e.g., a paragraph) and output a probability that the input term is a medical term. In addition to the input words, in some embodiments, the input to the neural network includes additional features that capture morphology. In various embodiments, such features include prefixes (e.g., “ab” or “anti”), suffixes (e.g., “suppression”) and other neighboring words of significance (e.g., “inhibitor,” “investigational,” or therapy).

In various embodiments, features may be extracted from words to represent linguistic similarities. In various embodiments, a cognitive (e.g., machine learning) model may be trained to predict medical versus non-medical words. In various embodiments, the cognitive model may extract features from words (e.g., length, part of words, endings, contextual features, etc.). In various embodiments, the features may be extracted as a feature vector. In various embodiments, the features may be input into a logistic regression model. In various embodiments, the features may be input into an artificial neural network, e.g., a long short-term memory (LSTM) network. In various embodiments, the output of the model(s) may be a prediction of whether the word is a medical term or not.

At 209, a metric is created in the medical term space for each specific condition. In some embodiments, a Map/Reduce cluster is used to compute the metric. Given this metric, a distance between two terms may be computed. The distance between two terms will be a vector of values, based on: the frequency they appear together in the condition corpus; the frequency they appear in proximity to a third medical term; their morphological resemblance. The morphological resemblance is scored based on a frequency analysis of the morphological structures and the number of their appearance in the corpus.

In various embodiments, for each category in the plurality of categories of clinical trials, the new medical term may be compared to each associated keyword to determine, and for each new medical term and associated keyword pair: (1) a metric space of the new medical term and associated keyword, (2) occurrences of the new medical term and associated keyword, and (3) occurrences of the new medical term, associated keyword, and an additional medical term. The metric space of a term and its associated keyword may be computed based on the difference between the metrics of each respective term, as described above. In various embodiments, a vector may be determined based on the above three components. In various embodiments, a vector magnitude may be determined for each vector representing a (new medical term, associated keyword) pair.

In various embodiments, the metric space of the new medical term and the associated keyword may be a distance metric between the new medical term and associated keyword. In various embodiments, the distance metric may be based at least in part on morphological similarity of the new medical term and associated keyword. In various embodiments, the distance metric may be based at least in part on semantic similarity of the new medical term and associated keyword. In various embodiments, the distance metric may be based at least in part on syntactic similarity of the new medical term and associated keyword. In various embodiments, the vector may include double occurrences and/or triple occurrences. In various embodiments, double occurrences comprise joint occurrences of the new medical term and associated keyword in the same clinical trial or clinical trial publication. In various embodiments, double occurrences comprise joint occurrences of the new medical term and associated keyword in the same category. In various embodiments, double occurrences comprise closeness of the new medical term and associated keyword in the same category. In various embodiments, triple occurrences comprise occurrences of the new medical term and associated keyword pair with an additional medical term. In various embodiments, triple occurrences comprise a number of different additional medical terms with which the new medical term and associated keyword pair has joint occurrence. In various embodiments, one or more smallest vector magnitude may be selected from the new medical term and associated keyword pairs.

In various embodiments, a vector of attributes may be used to represent each new term in the space (context) of the medical category. In various embodiments, the vector of attributes may include a linguistic breakdown of the term (e.g., part of word, prefix, suffixes, whether it includes other known terms as a subterm of this term, etc.), mentions and/or indices in external data sources (e.g., external medical data sources), and/or contextual features (e.g., does it usually comes with a number, type of treatment, etc.).

In various embodiments, the metric may be defined only for terms that have some similarities. In various embodiments, some of the terms (e.g., most or all) having a metric may be within the same field (e.g., medical). In various embodiments, the metric may be a representation of the term in the category space, i.e., a vector of attributes.

In various embodiments, a neural network language model may be used to generate distributed representations of texts in an unsupervised fashion, in the absence of deliberate feature engineering. In various embodiments, one neural network that may be used is Doc2Vec. The input of the neural network includes a sequence of observed words (e.g., “treatment of lymphoma”), each represented by a fixed-length vector, along with a text snippet token, also in the form of a dense vector and corresponding to the sentence/document source for the sequence. The concatenation or average of the word and paragraph vectors is used to predict the next word (e.g., “CD19”) in the snippet. In various embodiments, the two types of vectors may trained on any suitable number of paragraphs, for example, over 9,000 paragraphs. In various embodiments, training may be performed using stochastic gradient descent via backpropagation. At the testing stage, given an unseen paragraph, the word vectors are frozen from training time and the paragraph vector is inferred.

In various embodiments, the fixed length of the text feature vector m is a parameter in a Doc2Vec model. In various embodiments, since the length of the paragraphs is typically only two to three sentences, a short vector may be used. In various embodiments, this may also help limit the complexity of the transform network as it defines the number of output nodes. In an exemplary embodiment, m=10.

In various embodiments, another neural network that may be used is Word2Vec. The word2vec algorithm uses a neural network model to learn word associations from a large corpus of text. In various embodiments, once trained, a Word2Vecmodel can detect synonymous words or suggest additional words for a partial sentence. In various embodiments, a Word2Vec model represents each distinct word with a particular list of numbers called a vector. In various embodiments, the vectors may be chosen such that a simple mathematical function (e.g., the cosine similarity between the vectors) indicates the level of semantic similarity between the words represented by those vectors. In various embodiments, Word2Vec may include a group of related models that are used to produce word embeddings. In various embodiments, the Word2Vec models may be shallow, two-layer neural networks that are trained to reconstruct linguistic contexts of words. In various embodiments, Word2Vec may receive as its input a large corpus of text and produces a vector space, typically of several hundred dimensions, with each unique word in the corpus being assigned a corresponding vector in the space. In various embodiments, word vectors are positioned in the vector space such that words that share common contexts in the corpus are located close to one another in the space. In various embodiments, Word2Vec may utilize either of two model architectures to produce a distributed representation of words: continuous bag-of-words (CBOW) or continuous skip-gram. In various embodiments, in the continuous bag-of-words architecture, the model may predict the current word from a window of surrounding context words. In various embodiments, the order of context words does not influence prediction (bag-of-words assumption). In various embodiments, in the continuous skip-gram architecture, the model may use the current word to predict the surrounding window of context words. In various embodiments, the skip-gram architecture weighs nearby context words more heavily than more distant context words. In various embodiments, CBOW may be faster while skip-gram may be slower but does a better job predicting infrequent words. In various embodiments, high-frequency words often provide little information. In various embodiments, words with a frequency above a certain threshold may be subsampled to increase training speed. In various embodiments, high-frequency words may be removed. In various embodiments, quality of word embedding increases with higher dimensionality. In various embodiments, the dimensionality of the vectors may be set to between 100 and 1,000. In various embodiments, the size of the context window determines how many words before and after a given word would be included as context words of the given word. In various embodiments, a recommended value is 10 for skip-gram and 5 for CBOW.

In various embodiments, Word2Vec may be used to predict unknown or out-of-vocabulary (OOV) words and morphologically similar words, for example, in domains like medicine where synonyms and related words can be used depending on the preferred style of radiologist, and words may have been used infrequently in a large corpus. In various embodiments, if the Word2Vec model has not encountered a particular word before, it may use a random vector. In various embodiments, Intelligent Word Embedding (IWE) combines Word2Vec with a semantic dictionary mapping technique to handle information extraction from clinical texts, which include ambiguity of free text narrative style, lexical variations, use of ungrammatical and telegraphic phases, arbitrary ordering of words, and frequent appearance of abbreviations and acronyms. In various embodiments, an IWE model (trained on the one institutional dataset) may successfully translate to a different institutional dataset which demonstrates good generalizability of the approach across institutions.

In various embodiments, the use of different model parameters and different corpus sizes may affect the quality of a Word2Vec model. In various embodiments, accuracy can be improved in a number of ways, including the choice of model architecture (CBOW or Skip-Gram), increasing the training data set, increasing the number of vector dimensions, and/or increasing the window size of words considered by the algorithm. In various embodiments, each of these improvements comes with the cost of increased computational complexity and therefore increased model generation time. In various embodiments, in models using large corpora and/or a high number of dimensions, the skip-gram model may yield a higher overall accuracy, and produce (e.g., consistently) the highest accuracy on semantic relationships, as well as yielding the highest syntactic accuracy in most cases. In various embodiments, the CBOW may be less computationally expensive and yield similar accuracy results. In various embodiments, accuracy increases overall as the number of words used increases, and as the number of dimensions increases. In various embodiments, doubling the amount of training data may result in an increase in computational complexity equivalent to doubling the number of vector dimensions. In various embodiments, Word2vec may have a steep learning curve and may outperform another word-embedding technique (LSA) when it is trained with medium to large corpus size (e.g., more than 10 million words). In various embodiments, with a small training corpus, LSA may have better performance. In various embodiments, a best parameter setting may depend on the task and the training corpus. In various embodiments, for skip-gram models trained in medium size corpora, with 50 dimensions, a window size of 15 and 10 negative samples may be a suitable parameter setting.

In various embodiments, a text dataset is searched to find a closest match for a vector. In various embodiments, the closest match, or top few, in terms of Euclidean distance of text vector may be identified. In some embodiments, Mahalanobis distance is used in place of Euclidean distance. In various embodiments, vectors may be generated from input text by Doc2Vec and/or Word2Vec.

At 210, based on the condition metric, the distance of the new term from known medical terms is computed. The distance vector between each pair of terms is computed. Based on the distance, a semantic connection is established between new medical terms and disease categories. In particular, those terms which are close within the vector space are considered to have strong connections. Cluster analysis is applied to identify clusters of terms that represent medical concepts. This cluster of terms may be used for further analysis.

In various embodiments, the distance represents the semantic distance (e.g., similarity) between terms. In various embodiments, the distance may be between a known term and an unknown (e.g., new) term. In various embodiments, the known term may correspond to terms mapped to a particular disease condition. In various embodiments, the known term may correspond to an identified category of clinical trials. In various embodiments, for each new term, known terms associated with the new term may be determined, for example, using linguistic similarities, association through external semantic networks, and/or through co-mentions in the clinical trials corpus.

In various embodiments, the distance between two terms may be a distance function between the two vectors representing those terms. In various embodiments, the distance between two terms may include the co-mention of them in the trial corpus. In various embodiments, same terms may be used in different categories of clinical trials (representing different disease conditions). In various embodiments, the distance may be defined for each (category, term) pair. In various embodiments, the same term in different categories may have different distances to another term, depending on the category in which the determination is happening. In various embodiments, the vector(s) representing the pair(s) of terms having the smallest (i.e., minimum) distance may be selected. For example, for an identified new term, if vectors to 100 known terms associated with a particular disease condition are determined, the smallest vector may be selected and the unknown term mapped to the particular disease condition. In various embodiments, vector(s) may be selected such that the selected vectors represent a predetermined portion of all vectors. In various embodiments, the selected vectors may represented the smallest 1%, 2%, 3%, 4%, 5%, 6%, 7%, 8% 9%, 10%, 15%, 20%, etc. of vectors. In various embodiments, vector(s) may be selected such that the selected vectors are below a predetermined magnitude.

In various embodiments, for each pair of terms, a score may be determined representing the joined similarity of the pair. In various embodiments, the higher the similarity score, the smaller the distance between the terms.

In various embodiments, the semantic connection may be an edge in the graph between two terms that have close distance. In various embodiments, the threshold of what constitutes close for two terms may be configured manually. In various embodiments, if two terms are close enough, the two terms may be synonyms or not. In various embodiments, once the new term is semantically analyzed, clustering may be performed to cluster medical terms in medical criteria, to which the different clinical trials may be tagged. For example, out of 10K identified terms, there may be ˜1K criteria, and each trial may be tagged with ˜50 criteria.

Working Example: Clinical trial data is scraped from AACT using an API. Clinical trial NCT03488160 is identified and processed using an NLP algorithm. The trial contains the exclusion criteria “Prior treatment with idelalisib, other selective PI3Kδ inhibitors, or a pan-PI3K inhibitor.” The term “PI3Kδ inhibitors” is not recognized. Features are extracted from the term in question. This term is determined to likely be a new medical term based on a probability analysis. The term is determined to be similar in function and structure to “pan-PI3K inhibitor”, which is already known. The system creates a joined criteria that captures both terms and maps the new term as being related to “pan-PI3K inhibitor.” In various embodiments, the new term may also be mapped to the drug idelalisib. When looking at patient profiles, either terms may be used to match the patient to this particular trial.

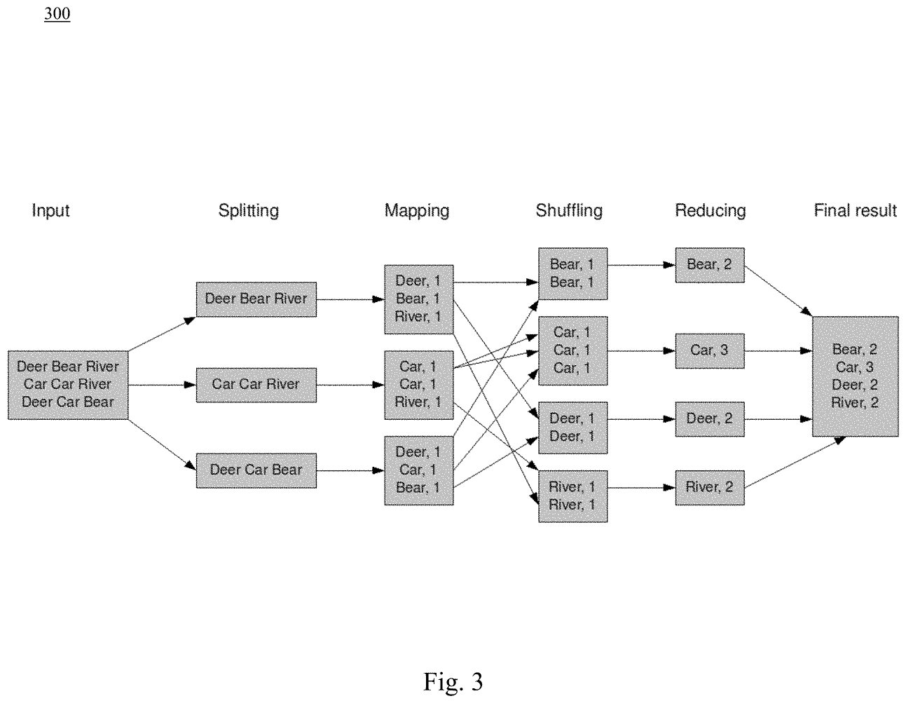

FIG. 3 depicts an exemplary Map/Reduce process 300 for counting words. Map/Reduce may be split into a mapping side and a reduce side. In particular, FIG. 3 illustrates the various steps in the Map/Reduce process, beginning with receiving input of text. In this particular example, the process 300 prepares intermediary key as pairs of (key,value) at a splitting stage where the key is the actual word and the value is the word's current frequency, namely 1 (thus splitting the text into three constituent parts). The process 300 then generates a count for each word in each constituent part and maps the words to a unique group of the same words at mapping and shuffling stages. The shuffling phase guarantees that all pairs with the same key will serve as input for only one reducer, so in the reduce phase, the frequency of each word can be calculated. The process 300 then reduces the instances of the words to a single instance (i.e., a single key) and increments the word count (i.e., increments the value). Lastly, the resulting key-value pairs are combined.

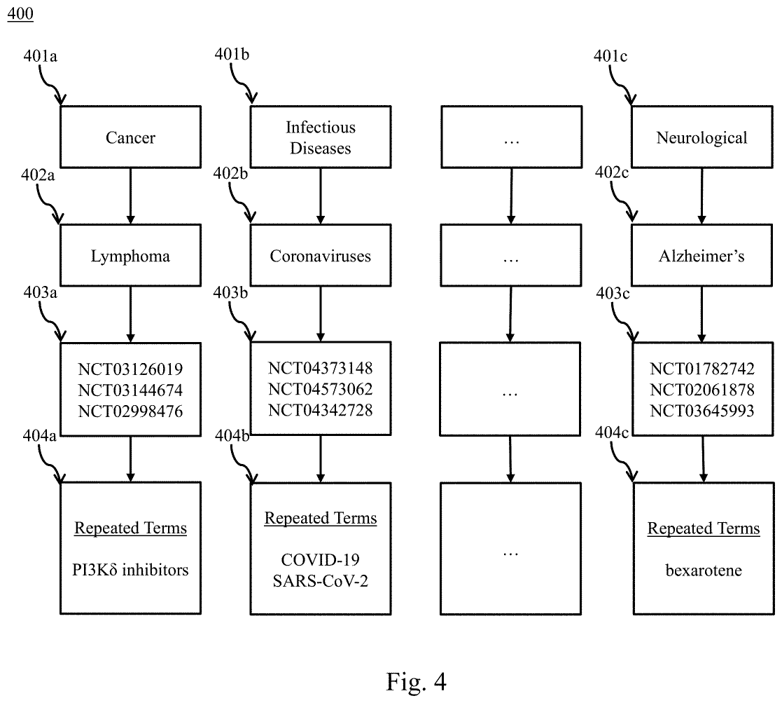

FIG. 4 depicts an exemplary process 400 for determining repeated words in categories of clinical trials. In various embodiments, clinical trial categories are determined based on a list of medical (e.g., disease) conditions. In various embodiments, the categorization process may include hierarchical categories. For example, FIG. 4 illustrates high-level categories of cancer 401a, infectious diseases 401b, and neurological 401c. FIG. 4 further illustrates lower-level categories of lymphoma 402a below cancer 401a, coronaviruses 402b below infectious diseases 401b, and Alzheimer's 402c below neurological 401c. The process 400 determines where each of one or more clinical trials should be categorized based on the medical condition list. In this example, three clinical trials 403a, 403b, 403c were found for each of these lower-level disease conditions. FIG. 4 shows that repeated terms 404a, 404b, 404c were identified from the clinical trial descriptions for the clinical trials 403a, 403b, 403c.

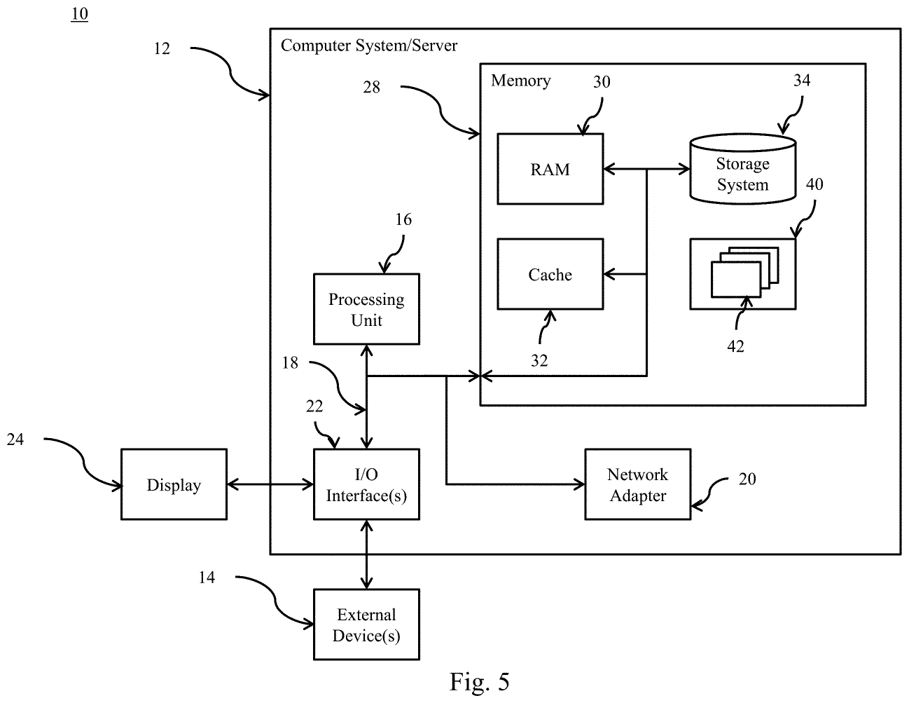

Referring now to FIG. 5, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method comprising:
reading one or more corpus, each of the one or more corpus comprising a plurality of clinical trial descriptions;
reading a list of disease conditions, the list having one or more disease condition mapped to one or more associated keyword;
determining a plurality of categories of clinical trials based on the plurality of clinical trial descriptions and the list of disease conditions;
determining a frequency of occurrence for each of one or more repeated terms from each category of clinical trials;
determining a set of category-specific repeated terms having a frequency of occurrence greater than a predetermined threshold;
determining whether each repeated term in the set of category-specific repeated terms is not present in a predetermined medical taxonomy to thereby identify a set of new terms;
determining a plurality of vectors for each new term in the set of new terms, each vector for a particular new term corresponding to the one or more associated keyword;
based on the plurality of vectors, selecting one or more of the new terms in the set of new terms; and
mapping each of the selected new terms to a disease condition thereby generating a supplemented list of disease conditions.

2. The method of claim 1, further comprising determining medical criteria from each of the plurality of clinical trial descriptions, wherein the medical criteria comprise inclusion criteria and/or exclusion criteria.

3. The method of claim 2, wherein determining medical criteria comprises applying an artificial neural network to the plurality of clinical trial descriptions.

4. The method of claim 2, further comprising:
reading a plurality of patient medical profiles;
determining one or more likely disease conditions for each patient medical profile based on the list of disease conditions;
determining one or more relevant patient medical profiles based on the determined medical criteria and the plurality of patient medical profiles;
for the relevant patient medical profiles, selecting one or more category of the plurality of categories of clinical trials based on the one or more likely disease conditions of the respective patient medical profile.

5. The method of claim 2, further comprising:
reading a plurality of patient medical profiles;
determining one or more likely disease conditions for each patient medical profile based on the supplemented list of disease conditions;
determining one or more relevant patient medical profiles based on the determined medical criteria and the plurality of patient medical profiles;
for the relevant patient medical profiles, selecting one or more category of the plurality of categories of clinical trials based on the one or more likely disease conditions of the respective patient medical profile.

6. The method of claim 1, wherein reading one or more corpus comprises accessing the one or more corpus via an application programming interface (API).

7. The method of claim 1, wherein the one or more keywords are unique for each disease condition.

8. The method of claim 1, wherein each of the plurality of categories of clinical trials corresponds to a unique disease condition.

9. The method of claim 1, wherein determining a frequency of occurrence comprises determining a score for each repeated term.

10. The method of claim 9, wherein the score represents a ratio of frequency of occurrence of the repeated word in its respective category of clinical trial to the frequency of occurrence in all other categories of clinical trials.

11. The method of claim 1, wherein the predetermined threshold is based on frequency of occurrence in a known medical taxonomy.

12. The method of claim 1, further comprising, for each new term in the set of new terms, determining a probability that the new term is a medical term.

13. The method of claim 1, further comprising determining a condition metric, wherein the plurality of distances are determined based in part on the condition metric.

14. The method of claim 13, wherein the condition metric is determined from a Map/Reduce cluster.

15. The method of claim 1, wherein each vector in the plurality of vectors comprises a frequency the particular new term and associated keyword appear together in the respective category of clinical trial, the frequency they appear in proximity to a third medical term, and the morphological resemblance between the particular new term and associated keyword.

16. The method of claim 15, wherein morphological resemblance is scored based on a frequency analysis of the morphological structures and the number of their appearance in the corpus.

17. The method of claim 1, wherein selecting the one or more new terms comprises determining one or more vectors of the plurality of vectors having a vector magnitude below a vector magnitude threshold.

18. The method of claim 1, wherein selecting the one or more new terms comprises determining one or more vectors of the plurality of vectors having a minimum vector magnitude.

19. (canceled)

20. A computer program product for supplementing a keyword mapping to disease conditions based on clinical trial descriptions, the computer program product comprising a computer readable storage medium having program instructions embodied therewith, the program instructions executable by a processor to cause the processor to perform a method comprising:
reading one or more corpus, each of the one or more corpus comprising a plurality of clinical trial descriptions;
reading a list of disease conditions, the list having one or more disease condition mapped to one or more associated keyword;
determining a plurality of categories of clinical trials based on the plurality of clinical trial descriptions and the list of disease conditions;
determining a frequency of occurrence for each of one or more repeated terms from each category of clinical trials;
determining a set of category-specific repeated terms having a frequency of occurrence greater than a predetermined threshold;
determining whether each repeated term in the set of category-specific repeated terms is not present in a predetermined medical taxonomy to thereby identify a set of new terms;
determining a plurality of vectors for each new term in the set of new terms, each vector for a particular new term corresponding to the one or more associated keyword;
based on the plurality of vectors, selecting one or more of the new terms in the set of new terms; and
mapping each of the selected new terms to a disease condition thereby generating a supplemented list of disease conditions.

21. A method comprising:
reading a plurality of categories of clinical trials, each category of clinical trials corresponding to a unique disease condition and having a plurality of associated keywords;
receiving a new medical term, wherein the new medical term is not present in any of the plurality of categories of clinical trials;
for each category in the plurality of categories of clinical trials, comparing the new medical term to each associated keyword to determine for each new medical term and associated keyword pair:
a distance metric between the new medical term and associated keyword;
double occurrence of the new medical term and associated keyword;
triple occurrences of the new medical term, associated keyword, and an additional medical term;
determining a vector magnitude for each new medical term and associated keyword pair.

22.-32. (canceled)
